(19) United States
(12) Patent Application Publication
(10) Pub. No.: US 2024/0307646 A1
(43) Pub. Date: Sep. 19, 2024

(54) CONNECTING SOCKET AND ROTARY SLEEVE FOR RESPIRATION

(71) Applicant: Loewenstein Medical Technology S.A., Luxembourg (LU)

(72) Inventor: Martin EIFLER, Glueckstadt (DE)

(21) Appl. No.: 18/675,314

(22) Filed: May 28, 2024

Related U.S. Application Data
(63) Continuation of application No. 15/621,024, filed on Jun. 13, 2017, now Pat. No. 12,023,444.

(30) Foreign Application Priority Data
Jun. 16, 2016 (DE) 102016007302.0

(51) Int. Cl. A61M 16/08; A61M 16/00; A61M 16/06; A61M 39/10

(52) U.S. Cl. CPC A61M 16/0816; A61M 16/06; A61M 16/0825; A61M 16/0875; A61M 39/10; A61M 16/00

(57) ABSTRACT

A respiratory device comprising a connecting socket which is inserted at least partially into a rotary sleeve and is latched thereto. The connecting socket and the rotary sleeve are disposed between a respiration hose and a patient interface, the connecting socket having an external diameter which is smaller than the internal diameter of the rotary sleeve. The rotary sleeve comprises on its inner side latching elements which engage in latching elements on the external diameter of the socket.

### CROSS-REFERENCE TO RELATED APPLICATIONS

The present application is a continuation of application Ser. No. 15/621,024, filed Jun. 13, 2017, which claims priority under 35 U.S.C. § 119 of German Patent Application No. 10 2016 007 302.0, filed Jun. 16, 2016. The entire disclosures of these applications are expressly incorporated by reference herein.

### BACKGROUND OF THE INVENTION

### 1. Field of the Invention

The present invention relates to a respiratory device configured as a patient interface. Respiratory devices are of importance to patients who suffer from a disturbance of the respiratory air or oxygen supply, wherein, via a patient interface which can be configured as a full-face mask, nose mask or nasal pillow mask, respiratory gas, or oxygen-enriched air, can be fed with a positive pressure support to the patient.

### 2. Discussion of Background Information

In order to connect the patient interface to the respiration hose, a rotary sleeve, which is connected via a releasable snap joint to a connecting socket of the mask, is generally used. This enables hose forces to be diverted through the use of a rotary function and allows the patient to easily separate the connection during a break in therapy.

The connecting socket is designed, for instance, as an elbow having a ball joint. The snap joint between the connecting socket and the rotary sleeve is generally realized by snap hooks in the connecting socket. For this, the connecting socket is of slotted design, so that resilient elements are obtained. These have the drawback that they can easily break off. As a result of the slotting, a small overlap between connecting socket and rotary sleeve is obtained, so that increased leaks occur.

A further problem is posed by the removal/release of the hose from the rotary sleeve. The hose coupling is much more firmly connected to the rotary sleeve than is the rotary sleeve to the elbow. For the release of the very tightly fitting connection of the hose coupling of the respiration hose from the rotary sleeve, only a very small holding or operating surface is available, which specifically for elderly patients or patients with restricted motility, is very difficult to handle.

In view of the foregoing, it would be advantageous to have available a device of the type stated in the introduction such that high functionality, improved handling and high reliability is provided.

### SUMMARY OF THE INVENTION

The present invention provides a respiratory device comprising or consisting of a connecting socket which is inserted at least partially into a rotary sleeve and is latched thereto. The connecting socket and the rotary sleeve are disposed between a respiration hose and a patient interface. Further, the connecting socket has an external diameter which is smaller than the internal diameter of the rotary sleeve, and the rotary sleeve has on its inner side latching elements, which engage in latching elements on the external diameter of the socket.

In one aspect, the socket may intrude with at least about 30%, e.g., at least about 60%, or at least 80% of its length L into the rotary sleeve.

In another aspect, the rotary sleeve may comprise on its inner wall a stop for the socket.

In yet another aspect, the respiration hose may comprise a hose coupling having an internal diameter D6 which is greater than the external diameter D5 of the rotary sleeve.

In a still further aspect, the rotary sleeve may be conical, so that the external diameter D5 of the rotary sleeve slowly widens up to the end facing the connecting socket.

In another aspect, the rotary sleeve may comprise a conical inner face.

In another aspect, the rotary sleeve may comprise symmetrical extensions (lugs) of the one-piece (unitary) outer wall.

In another aspect, the lugs may comprise on their inner wall an undercut or a latching portion, which serves for the latching connection to the socket.

In another aspect, the rotary sleeve may have an internal diameter D4 which is smaller than the external diameter D3 of the connecting socket.

In another aspect, the external diameter D3 of the connecting socket may widen conically into the external diameter D2.

In another aspect, the socket, for the latching to the rotary sleeve, may comprise a circumferential bevel or a step in the outer wall.

In another aspect, the socket may taper behind the step and may have the external diameter D1, which is smaller than D2 or D3.

In another aspect, the lugs may reach with the undercut over the step and thus may form a snap joint between rotary sleeve and socket.

In another aspect, an axial displacement between the rotary sleeve and the socket may be avoided by an end stop, against which the socket butts, in the rotary sleeve.

### BRIEF DESCRIPTION OF THE DRAWINGS

The following drawings illustrate the structure of a hose attachment to a patient interface. In the drawings:

FIG. 1 shows a connecting socket with rotary sleeve and respiration hose;

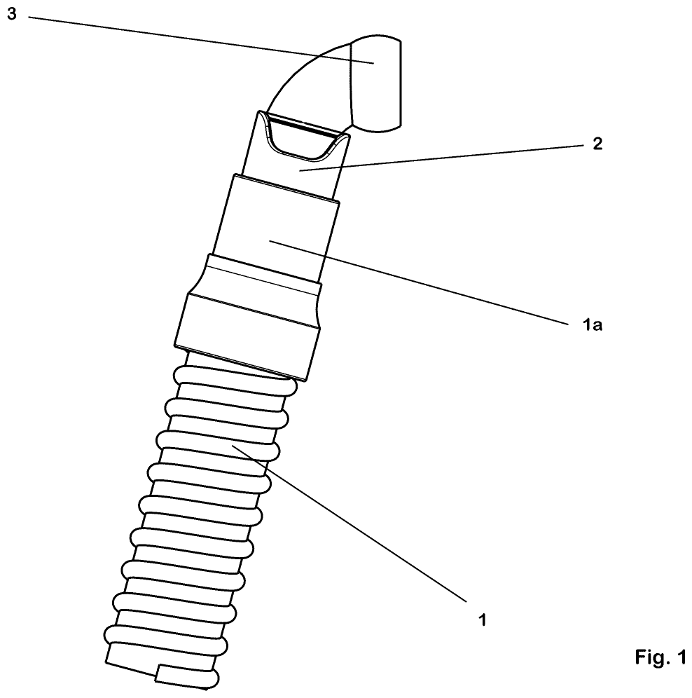

FIG. 2 is a sectional representation of the structure;

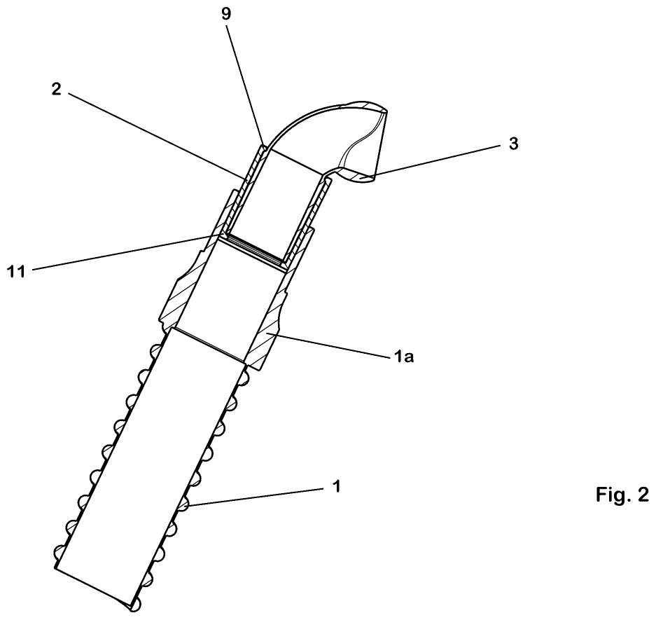

FIG. 3 is an exploded representation of the structure;

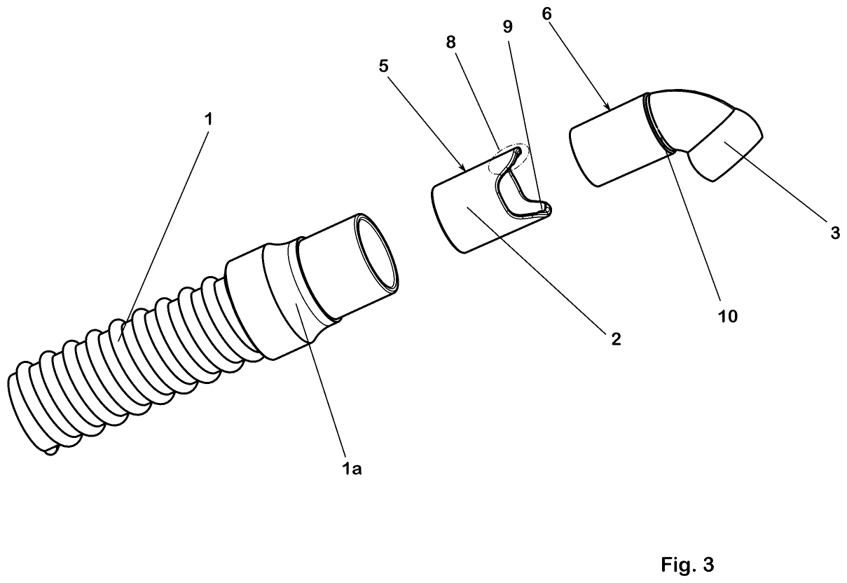

FIG. 4 shows the rotary sleeve connected to the connecting socket;

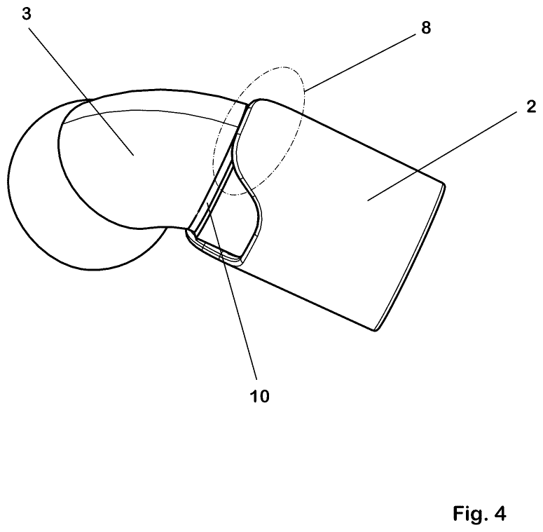

FIG. 5 is a sectional view through rotary sleeve and connecting socket;

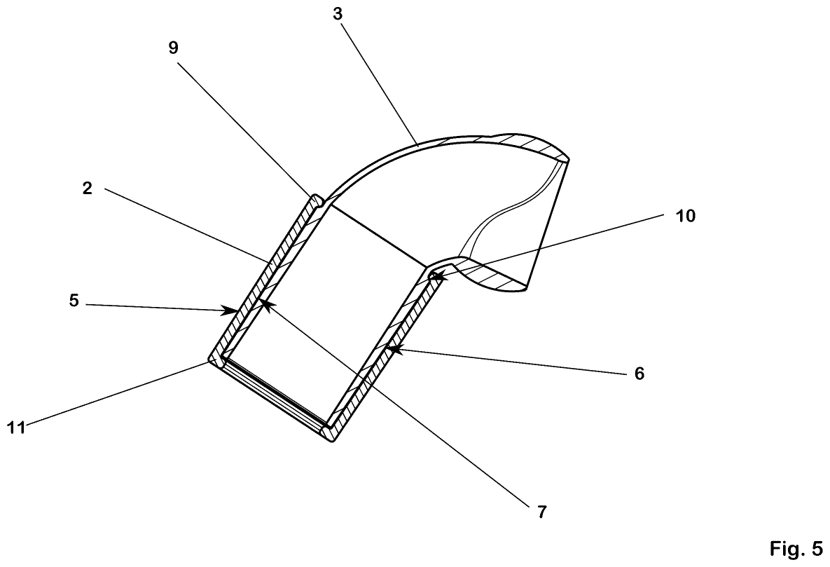

FIG. 6 shows a rotary sleeve.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

For the respiration, the hose (1), at whose ends is found a hose coupling (1a), is connected on the patient side, via a rotary sleeve (2) and a connecting socket (6), to the patient interface (4) (not represented). The hose coupling (1a) of the respiration hose (1) is slipped over the conically slightly thickening outer wall (5) of the rotary sleeve (2), to the point where it is tightly fitted. To this end, the hose coupling (1a) has an internal diameter D6 which is greater than the external diameter D5 of the rotary sleeve (2). The rotary sleeve has, for instance, a conically slightly thickening outer wall (5), so that the external diameter D5 of the rotary sleeve (2) slowly widens up to the end (8) facing the connecting socket.

The rotary sleeve (2) has, for instance, in addition to the conical outer face (5), also a conical inner face (7). This is configured so as to correspond with the socket (6) of the elbow connection (3) and, by virtue of a slight play, enables the smooth turning function of the components. The rotary sleeve (2) has symmetrical extensions (8) of the one-piece (unitary) casing, which can be referred to as lugs. The lugs (8) have an internally arranged undercut (9) or hook portion, which serves for the latching to the socket (6). The socket (6) has for the latching a circumferential bevel or a step (10) on the surface, behind which step the socket tapers slightly, with the external diameter D1. The rotary sleeve (2) has an internal diameter D4 which is smaller than the external diameter D3 of the connecting socket (6).

The lugs (8) reach with the undercut over the step (10) and thus form a snap joint between rotary sleeve (2) and socket (6). The connecting socket (6) has, for instance, a conical surface, which results in the external diameter D3 being smaller than the external diameter D2.

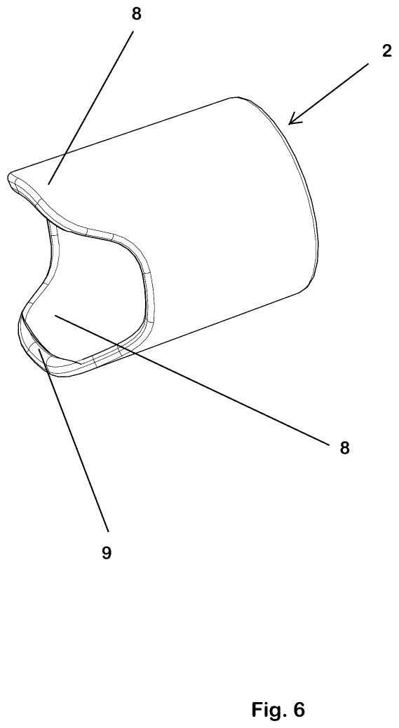

An axial displacement between the rotary sleeve (2) and the socket is avoided by an end stop (11), against which the socket (6) of the elbow connection (3) can maximally move, in the rotary sleeve (2). The details of the snap joint (undercut 9 and bevel 10), as well as the end stop (11), are evident in the sectional representation. The undercut (9) on the lugs (8) is represented in the detailed view, FIG. 6, of the rotary sleeve (2).

If the connection between hose (1) and mask (4) is to be broken, it is sufficient to pull on the hose coupling (1a) or the hose (1) and the snap joint between socket (6) and rotary sleeve (2) releases. The hose coupling (1a), however, still sits securely on the rotary sleeve (2). If, however, the connection between hose coupling (1a) and rotary sleeve (2) is to be released, for example for cleaning purposes, then the two extended lugs (8) additionally serve as holding or gripping surfaces. For the removal of the hose, the rotary sleeve is grasped via the lugs (8) and so simultaneously secures the snap joint-through the pressure on the lugs-when the hose (1) is pulled off the rotary sleeve (2) by slight bending of the hose coupling (1a).

As a result of the long, smooth overlap region between the rotary sleeve (2) and the socket, substantially fewer leaks occur than with the known, slotted snap joints. To this end, the connecting socket (6) is inserted at least with about 30% of its length, e.g., at least about 40% of its length, at least about 50% of its length, or at least about 60% of its length, into the rotary sleeve (2).

While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

### REFERENCE NUMERALS

1 respiration hose with

1a hose coupling

2 rotary sleeve

3 elbow connection with ball joint

4 respiration mask (not represented)

5 outer face of the rotary sleeve (2)

6 socket

7 inner face of the rotary sleeve (2)

8 lug

9 undercut

10 bevel, step

11 stop

What is claimed is:

1. A device for connecting a patient interface and a respiration hose, wherein the device comprises a connecting socket configured for being connected to the patient interface and a rotary sleeve configured to be connected to a respiration hose having a respiration hose coupling, the connecting socket being insertable at least partially into the rotary sleeve by having an external diameter which is smaller than an internal diameter of the rotary sleeve, the rotary sleeve comprising on its inner side latching elements that engage in latching elements on an external diameter of the connecting socket, the rotary sleeve having symmetrical extensions that are lugs comprised of a one-piece, unitary outer wall, and wherein for releasing a connection between the respiration hose coupling and the rotary sleeve without releasing a connection between the rotary sleeve and the connecting socket, the lugs can be pressed against the connecting socket to thereby secure the connection between the rotary sleeve and the connecting socket when pulling the respiratory hose coupling off the rotary sleeve.

2. The device of claim 1, wherein the connecting socket is insertable with at least 30% of its length into the rotary sleeve.

3. The device of claim 1, wherein the connecting socket is insertable with at least 60% of its length into the rotary sleeve.

4. The device of claim 1, wherein the connecting socket is insertable with at least 80% of its length into the rotary sleeve.

5. The device of claim 1, wherein the rotary sleeve comprises on its inner wall a stop for the connecting socket.

6. The device of claim 1, wherein the device further comprises a respiration hose coupling having an internal diameter which is greater than an external diameter of the rotary sleeve.

7. The device of claim 1, wherein the rotary sleeve has a conical inner face.

8. The device of claim 1, wherein the lugs comprise on their inner wall an undercut or latching portion which serves for the latching to the connecting socket.

9. The device of claim 1, wherein the external diameter of the connecting socket widens conically in a direction away from the rotary sleeve.

10. The device of claim 1, wherein the connecting socket, for latching to the rotary sleeve, comprises a circumferential bevel or a step in its outer wall.

11. The device of claim 10, wherein the connecting socket tapers behind the step and has an external diameter which is smaller than the external diameter of the connecting socket.

12. The device of claim 10, wherein the connecting socket tapers behind the step and has an external diameter which is smaller than a conically widened external diameter of the connecting socket.

13. The device of claim 8, wherein the lugs reach with the undercut over a step in the outer wall of the connecting socket to thereby form a snap joint between rotary sleeve and connecting socket.

14. The device of claim 1, wherein an axial displacement between the rotary sleeve and the connecting socket is prevented by an end stop in the rotary sleeve, against which end stop the connecting socket butts.

15. The device of claim 1, wherein the rotary sleeve has a conically slightly thickening outer wall so that the external diameter of the rotary sleeve widens up to the end that faces the connecting socket.

16. The device of claim 1, wherein two lugs are present.

17. A device for connecting a patient interface and a respiration hose comprising a respiration hose coupling, wherein the device comprises a connecting socket configured for being connected to the patient interface and a rotary sleeve configured to be connected to a respiration hose, the connecting socket being insertable at least partially into the rotary sleeve by having an external diameter which is smaller than an internal diameter of the rotary sleeve, the rotary sleeve comprising symmetrical extensions that are lugs comprised of a one-piece, unitary outer wall, which extensions have on their inner wall latching elements that engage in latching elements on an external diameter of the connecting socket, and wherein for releasing a connection between the respiration hose coupling and the rotary sleeve without releasing a connection between the rotary sleeve and the connecting socket, the lugs can be pressed against the connecting socket to thereby secure the connection between the rotary sleeve and the connecting socket when pulling the respiratory hose coupling off the rotary sleeve.

18. The device of claim 17, wherein the device further comprises a respiration hose coupling having an internal diameter which is greater than an external diameter of the rotary sleeve.

19. The device of claim 17, wherein the lugs comprise on their inner wall an undercut or latching portion which serves for the latching to the connecting socket.

20. The device of claim 17, wherein the external diameter of the connecting socket widens conically in a direction away from the rotary sleeve.
